(19) United States
(12) Patent Application Publication
(10) Pub. No.: US 2011/0245606 A1
(43) Pub. Date: Oct. 6, 2011

(54) ENDOSCOPIC GASEOUS MATERIAL FEED SYSTEM

(76) Inventors: Kentaro HAYASHI, Kanagawa (JP); Mutsumi NARUSE, Kanagawa (JP)

(21) Appl. No.: 13/015,926

(22) Filed: Jan. 28, 2011

(30) Foreign Application Priority Data
Mar. 31, 2010 (JP) 2010-081713

(51) Int. Cl. A61B 1/015; A61B 1/045

(52) U.S. Cl. 600/109; 600/158

(57) ABSTRACT

An endoscopic gaseous material feed system comprises a gas feeder 3 having a gas tank 60 containing carbon dioxide gas, a light source 2 incorporating an air pump 23 for air supply, an endoscope 1 internally having a gaseous material passage for a gas or air supply to a body cavity of a patient or examinee, and a controller 80 adapted to put the air pump 23 of the light source 2 in a deactivated state upon detection of a start of a carbon dioxide gas supply from the gas feeder 3.

### FIELD OF THE INVENTION

This invention relates to an endoscopic gaseous material feed system for medical use, having a gas feeder to supply carbon dioxide gas to an endoscope which is connected to a light source having an air supply source.

### BACKGROUND OF THE INVENTION

In endoscopy, air or a gas, as a gaseous material, is supplied to a body cavity of a patient or examinee for the purpose of securing a view field of an endoscope or for securing a space which is required for manipulation of a surgical or biopsy tool as an unlimited source of gaseous material. Heretofore, it has been the general practice to use air as a gas to be delivered to a body cavity. However, instead of air, carbon dioxide gas (CO₂) is increasingly used for this purpose, in consideration of better in vivo absorption and less damages to a patient or examinee.

Air is delivered to a body cavity from an air pump which is provided internally of a light source enclosure. On the other hand, carbon dioxide gas is delivered from a gas tank which is packed with carbon dioxide gas and replaceably loaded on a gas feeder. A gas feed system having such light source and gas feeder is disclosed in Japanese Laid-Open Patent Application 2006-14961. According to this technology, a gas feeder and a light source are connected with each other by way of a gas tube to supply an endoscope selectively with air from an air pump or carbon dioxide gas from the gas feeder through a control mechanism provided inside the light source.

In this case, a selector switch is provided on an operation panel on the light source thereby to make a selection between air and carbon dioxide gas in starting a gas supply to a body cavity, controlling the connected light source and gas feeder to effectuate a supply of a selected gas. The light source and the gas feeder are communicable with each other by way of a communication cable. When air is selected by the selector switch, an electromagnetic valve of the gas feeder is closed to make a supply of carbon dioxide gas infeasible, while the air pump is put in operation for an air supply. On the other hand, when carbon dioxide gas is selected, the above-mentioned electromagnetic valve is opened to start a supply of carbon dioxide gas, while the air pump is held in a deactivated state.

The light source and gas feeder are separate units and separately turned on and off by the gas feed system. The light source, i.e. an illumination light source, is always kept on as long as an endoscopic examination or treatment is underway, irrespective of activation and deactivation of the air pump. That is, without exception, the power source of the light source is always turned on at the time of an endoscopic examination or treatment. On the other hand, for a carbon dioxide gas supply, the power source of the gas feeder is turned on to supply carbon dioxide gas from the gas tank. At this time, the light source is left on.

Within an enclosure of the light source, an air supply route and a gas supply route are joined together. That is to say, there is a possibility that air and carbon dioxide gas are simultaneously put in supply by an erroneous operation although it is a requisite to supply a selected gas alone. In Japanese Laid-Open Patent Application 2006-14961 mentioned above, arrangements are made to preselect one of the two feed gaseous materials by way of a selector switch. Therefore, at the time of switching the feed gas from air to carbon dioxide gas, it becomes necessary to turn on the power source of the gas feeder and at the same time to depress the selector switch for a switch to carbon dioxide gas, thus involving two kinds of switching operations for a switch of the feed gaseous material.

In case a selector switch is provided on a light source as in Japanese Laid-Open Patent Application 2006-14961, it becomes necessary to operate two different switches at two different locations. Especially, it is extremely troublesome to operate two different switches on the light source and gas feeder which are mounted separately on two carts or mounted jointly on one and same cart, one on the upper side of the other, as shown in FIG. 20 of Japanese Laid-Open Patent Application 2006-14961.

### SUMMARY OF THE INVENTION

With the foregoing in view, it is an object of the present invention to provide an endoscopic gaseous material feed system, which is capable of switching a feed gaseous material between carbon dioxide gas and air in such a way as to lessen burdens on the part of an operator.

In order to achieve the above-stated objective, according to the present invention, there is provided an endoscopic gaseous material feed system, comprising: a gas feeder having a gas tank packed with carbon dioxide gas; a light source incorporating an air pump for air supply; an endoscope internally provided with a gaseous material passage to supply carbon dioxide gas or air to a body cavity of a patient or examinee; and a controller adapted to hold the air pump of the light source in a deactivated state upon detection of a start of carbon dioxide gas supply from the gas feeder.

According to this endoscopic gaseous material feed system, the controller is adapted to take preference of a carbon dioxide gas, as one of gaseous material, supply from the gas feeder over an air, as another gaseous material, supply from the light source when a supply of whichever gas is feasible. Since the air pump is automatically in put in a deactivated state upon starting a supply of carbon dioxide gas, the operator has no need for manually turning off the air pump. Thus, the feed gas can be switched in such a way as to lessen burdens on side of an operator.

In a preferred form of the invention, the endoscopic gaseous material feed system further comprises a processor which is adapted to perform video signal processing operations to produce video signals of endoscopically captured picture images on the basis electric signals from a solid-state image sensor device provided at a fore distal end of the endoscope, and the above-mentioned controller is incorporated into the processor which is electrically connected with the gas feeder and the light source.

In this instance, the controller is incorporated into the processor, which is electrically connected with the light source having an air pump to be put in operation for an air supply, as well as with the gas feeder to be put in operation for a carbon dioxide gas supply. On the other hand, the processor usually incorporates a processor unit which is capable of performing complicate signal processing operations. Therefore, such a processor unit can be utilized to perform the functions of the above-mentioned controller of the gaseous material feed system. That is to say, the controller of the gaseous material feed system can be realized by reconstructing existing gas feeder and light source without adding a complicate construction.

In another preferred form of the invention, the controller is adapted to suspend a carbon dioxide gas supply from the gas feeder upon detection of residual gas content in the gas tank dropping below a predetermined threshold value, while activating the air pump of the light source to start a supply of air.

According to this endoscopic gaseous material feed system, carbon dioxide gas is preferentially used as a feed gas but its source (a gas tank) has a limit and may become deficient. Therefore, when residual gas content in the gas tank drops below a predetermined threshold value, the gas feed is switched to air to avoid an interruption of an ongoing endoscope examination or treatment.

In another preferred form of the invention, the endoscope is internally provided with a gas feed conduit connected to a junction of a carbon dioxide gas supply route from the gas feeder and an air supply route from the light source, a liquid feed conduit adopted as a passage of a liquid delivered from a liquid feed tank under pressure applied by the carbon dioxide gas or air on a liquid surface in the liquid feed tank, and a fluid feed valve adapted to selectively take in either a feed gas delivered under pressure through the gas feed conduit or a feed liquid delivered under pressure through the liquid feed conduit; the fluid feed valve being adapted to permit replacement of an open-to-atmosphere type fluid feed valve in communication with the atmosphere for release of a gas in the gas feed conduit, by a closed-to-atmosphere type fluid feed valve blocking communication of the gas feed conduit with the atmosphere, or vice versa.

In this case, the fluid feed valve is replaceable by a different type. At the time of feeding carbon dioxide gas, a closed-to-atmosphere type fluid feed valve is set on the endoscope to prevent wasteful consumption of carbon dioxide gas. On the other hand, at the time of feeding air, an open-to-atmosphere type fluid feed valve is set on the endoscope to release an excessively elevated pump pressure. Therefore, it is made possible to choose a fluid feed valve between two different types to carry out an endoscopic examination or treatment in a favorable manner.

In a preferred form of the invention, the endoscopic gaseous material feed system further comprises a relief mechanism for the purpose of relieving pump pressure of the air pump to the atmosphere when reached beyond a predetermined upper limit.

In this case, an excessive buildup of pressure is released to the atmosphere by the relief mechanism. A buildup of an excessively high pressure can occur when the air pump is erroneously put in operation during a closed-to-atmosphere type fluid feed valve is set on the endoscope. On such an occasion, an excessively high pressure is automatically released into the atmosphere through the relief mechanism.

In another preferred form of the invention, the endoscopic gaseous material feed system further comprises a main power switch thereby to turn on and off the gas feeder and said light source together, the controller being adapted to start a carbon dioxide gas supply from the gas feeder when the main power switch is turned on, while suspending an air supply from the air pump.

In this case, by the provision of the main power switch which turns on the two gas sources together, the endoscopic gaseous material feed system can be put in a position to start a supply of carbon dioxide gas preferentially over air. That is to say, simply by turning on the main power switch, the gaseous material feed system becomes ready to start a supply of carbon dioxide gas to a body cavity.

As described above, from the standpoint of lessening damages to a patient or examinee, the endoscopic gaseous material feed system according to the present invention is adapted to take preference of a carbon dioxide gas supply from a gas feeder over an air supply from an air pump of a light source. At the time of switching the gas feed from air to carbon dioxide gas, the air pump is put in a deactivated state in synchronism with a start of supply of carbon dioxide gas. Thus, the gas feed can be automatically switched from air to carbon dioxide gas without necessitating to manually turning off the air pump. This means that the system according to the invention is particularly arranged to relieve an operator of troublesome manual switching operations, with a view to lessening burdens on the part of the operator.

### BRIEF DESCRIPTION OF THE DRAWINGS

In the accompanying drawings:

FIG. 1 is a schematic view, showing a general layout of an endoscopic gaseous material feed system embodying the present invention;

FIG. 2 is a schematic sectional view of a closed-to-atmosphere type fluid feed valve;

FIG. 3 is a block diagram showing configurative relations of light source, processor and gas feeder; and

FIG. 4 is a schematic sectional view of a closed-to-atmosphere type fluid feed valve.

### DESCRIPTION OF PREFERRED EMBODIMENTS

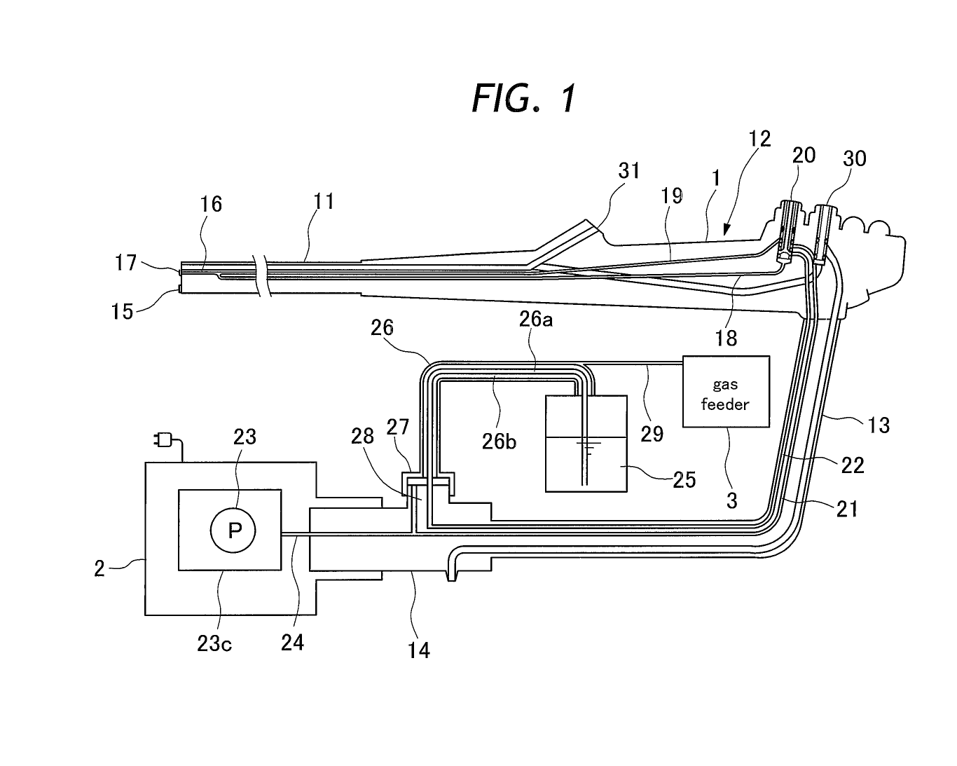

Hereafter, the invention is described more particularly by way of its preferred embodiments. Needless to say, the present invention should not be construed as being limited to particular examples shown. FIG. 1 schematically shows a general layout of a gaseous material feed system according to the present invention, which is largely constituted by an endoscope 1, a light source 2, a gas feeder 3 and a processor 4 as described in greater detail below. The endoscope 1 is introduced into a body cavity for the purpose of medical examinations or treatments. Examples of the endoscope 1 include not only flexible endoscopes in use in upper or lower endoscopy but also rigid type endoscopes like a laparoscope.

For instance, the endoscope 1 is used to give a medical treatment in an endoscopic surgical operation. The light source 2 mainly serves as an illumination light source and incorporates an air pump in its enclosure to supply air to a body cavity. The gas feeder 3 is a carbon dioxide (CO₂) gas source from which carbon dioxide gas is supplied to a body cavity. The processor 4 is adapted to perform video signal processing operations for endoscopically captured picture images.

The endoscope 1 is largely composed of an elongated insertion rod 11, a manipulating head assembly 12 and a universal cable 13. The insertion rod 11 is so shaped as to permit introduction into a body cavity of a patient or examinee under control of manipulating and operating mechanisms on the manipulating head assembly 12 to be gripped by an operator. A light source connector 14 at a proximal end of the universal cable 13 is disconnectibly connected to the light source 2. An illumination window (not shown) and an observation window 15 are provided on a fore distal end portion of the insertion rod 11 to capture the observation window 15 images of an intracavitary site under illumination by light projected through the illumination window. A camera unit having an objective lens and a solid-state image sensor device is fitted inside the observation window 15.

The endoscope 1 is internally provided with a fluid feed mechanism. A liquid is supplied through the endoscope, for example, at the time of washing the observation window 15 when contaminated with body fluids, or at the time of cleaning intracavitary wall surfaces prior to an examination or for the purpose of irrigative cleaning. On the other hand, a gas is supplied to inflate a body cavity or to dissipate liquid droplets from the observation window 15 after washing. In endoscopes in general, a fluid feed system is adapted to supply water and air to a body cavity.

In this regard, the endoscope 1 is provided with a fluid supply passage 16 internally through the insertion rod 11, the fore distal end of the passage 16 being formed into the shape of a jet nozzle 17 which is directed toward the observation window 15. This fluid supply passage 16 in the insertion rod 11 is bifurcated into a gas supply passage 18 and a liquid supply passage 19 on its way and extended into the manipulating head assembly 12. The gas supply passage 18 and liquid supply passage 19 are connected to a fluid feed valve 20 which is provided internally of the manipulating head assembly 12. The fluid feed valve 20 is connected with a gas feed conduit 21 and a liquid feed conduit 22 which are brought into and out of communication by operating the fluid feed valve 20.

A gas supply route is constituted by the above-mentioned gas supply passage 18 and gas feed conduit 21, while a liquid supply route is constituted by the above-mentioned liquid supply passage 19 and liquid feed conduit 22. Thus, the fluid supply passage 16 forms a common terminal passage for the gas supply passage and the liquid supply passage to supply compressed air or a cleaning liquid selectively to the jet nozzle 17. The gas feed conduit 21 and the liquid feed conduit 22 are extended into the universal cable 13 from the manipulating head assembly 12, and led to the light source connector 14.

An illumination lamp (not shown) is housed in the light source 2 as a source of illumination light. By way of a light guide, illumination light from this lamp is transferred as far as an endoscopic observation means which is provided on a fore distal end portion of the insertion rod 11. Thus, illumination light is projected through the illumination window. Further, an electric connector (not shown) is branched out from the universal cable 13 and disconnectibly connected to the processor 4.

An air pump 23 is built in the light source 2 to serve as a source of compressed air under control of a pump control block 23c. Connected to the air pump 23 is a compressed air pipe 24 through which compressed air is delivered by operation of the air pump 23. The liquid supply passage 19 serves as a cleaning liquid supply route to supply a cleaning liquid from a liquid feed tank 25. This liquid feed tank 25 is located outside the light source 2. A double tube conduit 26 is connected to the liquid feed tank 25, the double tube conduit 26 including an inner tube serving as a cleaning liquid conduit 26a and an outer tube serving as a pressurization conduit 26b. A piping connector member 27 which is attached to the fore distal end of the double tube conduit 26 is disconnectibly connected to a piping connector member 28 which is provided on the side of the light source connector 14.

Of the double tube conduit 26, one end of the cleaning liquid conduit 26a is immersed in a cleaning liquid in the liquid feed tank 25, while one end of the pressurization conduit 26b is opened into the tank 25 at a position above the surface of the cleaning liquid. A gas feed conduit 29 which is connected from the gas feeder 3 as a carbon dioxide gas supply route is connected to the pressurization conduit 26b. Thus, the pressurization conduit 26b is bifurcated at a proximal end, one of the bifurcated ends being connected to the gas feed conduit 29 and the other end being led to the liquid feed tank 25. The pressurization conduit 26b is connected to the gas feed conduit 29 and to a pressurization air conduit 24 in the light source connector 14 to receive compressed air from the air pump 23. Thus, a pressure is applied to the liquid surface in the liquid feed tank 25 by introducing carbon dioxide gas or air through the pressurization conduit 26b which is exposed at an upper portion of the tank 25.

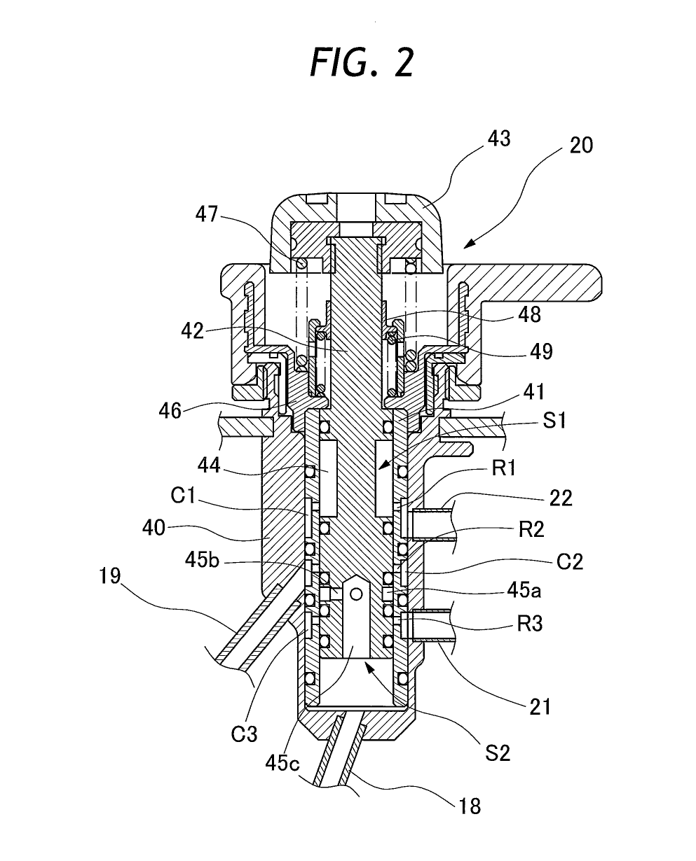

A fluid feed valve 20 and a suction valve 30 are provided on the manipulating head assembly 12, along with a tool entrance way 31 for introduction of a surgical or biopsy tool. As exemplified in FIG. 2, the fluid feed valve 20 is adapted to be manipulated by a finger of a hand which grips the manipulating head assembly 12, for selectively supplying a gas or liquid toward the jet nozzle 17. For this purpose, a valve casing 40 is provided on the manipulating head assembly 12 to accommodate the fluid feed valve 20 which is connected to the gas supply passage 18 and the liquid supply passage 19 on the side of the insertion rod 11 and to the gas feed conduit 21 and the liquid feed conduit 22 on the side of the gas and liquid sources as well. A valve guide 41 is fitted in the inner periphery of the valve casing 40, and in turn a valve member 42 is axially movably fitted in the valve guide 41. A switch button 43 is attached to the outer end of the valve member 40 in such a way as to project on the outer side of the valve casing 40.

The valve member 42 functions to open and close communications between the gas supply passage 18 and the gas feed conduit 21, and between the liquid supply passage 19 and the liquid feed conduit 22. For this purpose, a first annular chamber C1, a second annular chamber C2 and a third annular chamber C3 are provided around the outer periphery of the valve guide 41. The liquid feed conduit 22 is connected to the first annular chamber C1, the liquid supply passage 19 is connected to the second annular chamber C2, and the gas feed conduit 21 is connected to the third annular chamber C3. Further, the gas supply passage 18 is opened into the bottom of the valve casing 40, which is not covered with the valve guide 41.

Communication passages R1 to R3 are bored axially through the annular chambers C1 to C3, respectively. Further, the valve member 42 is provided with a first switch portion S1 to switch on and off communications between the liquid supply passage 19 and the liquid feed conduit 22, along with a second switch portion S2 to switch on and off communications between the gas supply passage 18 and the gas feed conduit 21. The first switch portion S1 is constituted by a first annular passage 44 which is formed around the outer periphery of the valve member 42; and the second switch portion S2 is constituted by a second annular passage 45a formed under the first annular passage 44, a communication hole 45b bored axially through the valve member 42, and an axial passage 45c opened in the bottom of the valve member 42.

In the position shown in FIG. 2, communication between the gas supply passage 18 and the gas feed conduit 21 as well as communication between the liquid supply passage 19 and the liquid feed conduit 22 is blocked. If the switch button 43 is pushed in from that position to lower the valve member 42, the gas supply passage 18 and the gas feed conduit 21 are brought into communication with each other through the first switch portion 21 in the course of a downward stroke. However, in this state, communication between the liquid supply passage 19 and the liquid feed conduit 22 is still blocked. That is to say, the fluid feed valve 20 is now switched to a gas feed position. As the switch button 43 is pushed in further toward a liquid feed position, communication between the gas supply passage 18 and the gas feed conduit 21 is blocked, and the liquid supply passage 19 and the liquid feed conduit 22 are brought into communication with each other through the first switch portion S1.

A return spring 47 is interposed between the valve member 42 and a spring holder 46 which is provided on the valve casing 40 to bias the valve member 42 toward an outermost initial position. Further, a spring holder 48 is attached on the valve member 42, and a second spring member 49 is interposed between this spring holder 48 and the spring holder 46 to give a feeling of a stepwise switching action. Therefore, when no external force is applied, the switch button 43 is retained in the initial blocking position under the influence of the biasing action of the return spring 47. Upon pushing the switch button 43 inward against the action of the return spring 47, the valve member 42 is axially slid along the valve guide 41, switching the valve 20 to a gas feed position at a middle point of the inward stroke of the valve member 42. Until this gas feed position is reached, no biasing force is applied to the valve member 42 from the second spring 49. As soon as the valve member 42 is switched to the gas feed position, the lower side of the switch button 43 is abutted against the spring holder 48. Therefore, in order to further push down the valve member 42 from this point, the switch button 43 needs to be pushed in against a biasing force which increased by superposition of a biasing force of the second spring 49. That is to say, by pushing down the switch button 43 further against joined biasing forces of the return spring 47 and the second spring 49, the valve member 42 is switched to a liquid feed position.

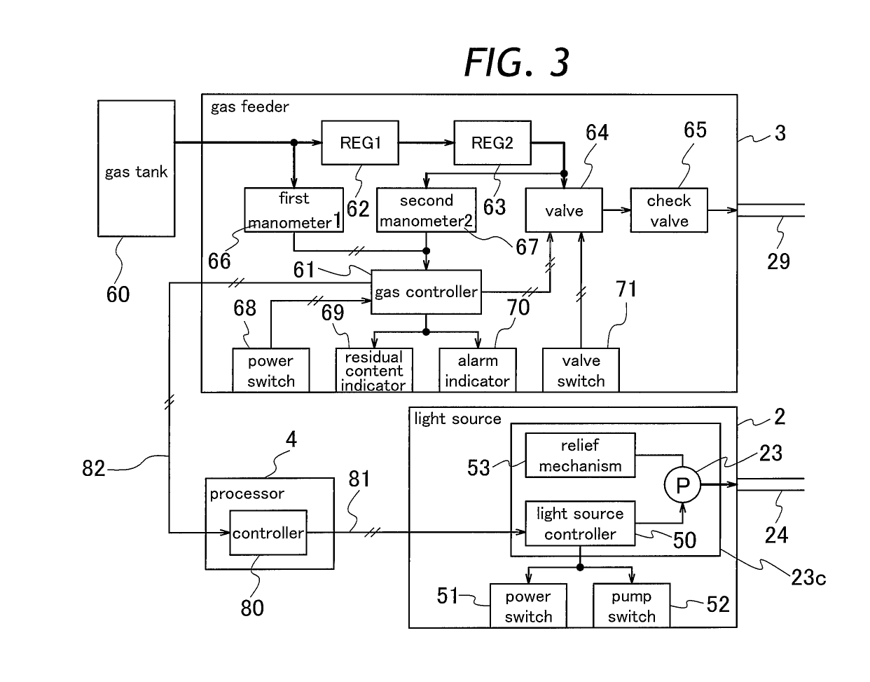

The present invention is not limited to the particular endoscopic fluid feed mechanism shown in the drawing. In the particular example shown, as a liquid, a cleaning liquid is supplied from the liquid feed tank 25. On the other hand, as a gas, basically carbon dioxide gas is supplied through the mechanism. However, if necessary, air is supplied from the air pump 23 which is built in the light source 2. For this purpose, the present invention adopts an endoscopic gaseous material feed system as shown in FIG. 3. In this instance, in addition to the air pump 23, the light source 2 is comprised of a light source controller 50, a power switch 51, a pump switch 52 and a relief mechanism 53. By operation of the pump 23, a pump pressure comes into effect to supply compressed air to the pressurization conduit 24. The light source controller 50 at the control of the air pump 23 is adapted to make a decision as to whether or not activate the air pump 23 in consideration of operating conditions.

The power switch 51 serves to turn on and off a power supply to the light source 2. When the switch 51 is off, not only the air pump 23 but also the supply of illumination light is turned off. The pump switch 52 is provided to activate or deactivate the air pump 23. Even when this switch 52 is off, the supply of illumination light is kept on as long as the light source 2 is on. The light source controller 50 is connected to the power switch 51 and the pump switch 52 to control the operation of the air pump 23. The light source controller 50 is adapted to turn on the air pump 23 automatically when the power switch 51 is turned on, without necessitating turning on the pump switch 52 separately.

The gas feeder 3 is loaded with a gas tank 60, and largely constituted by a gas controller 61, a first regulator (REG1) 62, a second regulator (REG2) 63, a valve 64, a check valve 65, a first manometer (manometer 1) 66, a second manometer (manometer 2) 67, a power switch 68, a residual content indicator 69, an alarm indicator 70 and a valve switch 71. The gas tank 60 is packed with carbon dioxide gas to serve as a source of carbon dioxide gas, and, when emptied up or at a suitable timing, it can be dismantled and replaced by a full one.

The gas controller 61 is at the control of the gas feeder 3 as a whole, including control of the power source or supply. The first regulator 61 is connected to the gas tank 60 to reduce the pressure of carbon dioxide gas from the gas tank 60. Since carbon dioxide gas from the gas tank 60 is at a high pressure level, it is reduced by two steps. That is to say, after a pressure reduction at the first regulator 62, carbon dioxide is passed through the second regulator 63 for a second pressure reduction. The valve 64 is connected between the gas feed conduit 29 and the second regulator 63 through the check valve 65, and opened and closed to establish or cut off communication with the gas feed conduit 29. As for the valve 64, there may be employed an electromagnetic valve which is capable of on-off control by energization and de-energization of a solenoid. The check valve 65 is provided to block an inverse gas flow from the side of the gas feed conduit 29.

The first manometer 66 plays the role of detecting the pressure of carbon dioxide gas from the gas tank 60, while the second manometer 67 plays the role of detecting the pressure of carbon dioxide gas after a pressure reduction by the second regulator 63. Detected gas pressures are output to the gas controller 61 which is adapted to recognize a residual content of the gas tank 60 on the basis of a gas pressure of the tank 60. The power switch 68 is provided to turn on and off the power supply to the gas feeder 3. The residual content indicator 69 is adapted to indicate residual gas content as recognized by the gas controller 61. The alarm indicator 71 is adapted to indicate an alarm signal or message in the event an abnormal pressure is detected by the first manometer 66 or second manometer 67. If desired, the alarm indicator 71 may be arranged to give off an alarm sound to draw attention. The valve switch 71 is connected to the valve 64 to manually open and close the latter. Basically, the valve 64 is opened and closed under the control of the gas controller 61 but can be manually opened and closed by means of the valve switch 71.

The processor 4 performs video signal processing operations to generate video signals of endoscopically captured picture images, on the basis electric signals of the solid-state image sensor device on an endoscopic observation means at the fore distal end of the insertion rod 11. Basically, the processor 4 is configured to process video signals of endoscopically captured picture images, but includes a controller 80 in control of the light source controller 50 and the gas controller 61. For this purpose, the light source 2 and the gas feeder 3 are connected with each other by way of the processor 4, and the controller 80 is connected with the light source controller 50 and the gas controller 61 by the first and second communication lines 81 and 82. Further, the controller 80 in the processor 80 is adapted to control the light source 2 and the gas feeder 3.

With the arrangements as described above, at the start of an endoscopic examination or treatment using the endoscope 1, at least illumination light needs to be fed to the endoscope 1 from the light source 2. Therefore, in the first place, the power switch 51 is turned on to supply illumination light from the light source 2. Besides, in this case, the air pump 23 is automatically started when the power switch 51 is turned on. As a consequence, compressed air is automatically supplied to the endoscope 1 as soon as the light source 2 is turned on. However, if desired, arrangements may be made to start the pump 23 afterwards by a manual operation on the pump switch 52, instead of automatically starting same when the power switch 51 of the light source 2 is turned on.

There are two feed gas sources, including the air pump 23 of the light source 2 and carbon dioxide gas in the gas tank 60 of the gas feeder 3. According to the present invention, from the standpoint of lessening damages to a patient or examinee, the gas feed system is arranged to take preference of a carbon dioxide gas supply over an air supply. However, in consideration of an operator or operators who are more accustomed to operations for an air supply from the light source 2 than operations for a carbon dioxide gas supply from the gas feeder 3, the gas feed system is arranged to permit an air supply although priority is given to a carbon dioxide gas supply.

The gas feeder 3 is put in operation when the power switch 68 is turned on by an operator. At this time, the turning-on of the switch 68 is detected by the gas controller 61, and a switch-on notice is sent to the controller 80 of the processor 4 via the second communication line 82. Upon receiving the switch-on notice, the controller 80 controls the light source controller 50 of the light source 2 via the first communication line 81 to turn off the air pump 23.

As described hereinbefore, the air pump 23 is started when the light source 3 is turned on. Otherwise, the air pump 23 may have been put in operation manually by way of the pump switch 52. Therefore, the air pump 23 needs to be turned off to suspend an air supply. However, in case the air pump 23 is not activated automatically at the start or in case the air pump 23 is left in a deactivated state by depression of the pump switch 52, the light source controller 50 simply maintains the air motor 23 in a deactivated state regardless of a control signal from the controller 80 of the processor 4.

On the other hand, the gas controller 61 notifies the turning-on of the power switch 68 and opens the valve 64. As a consequence, carbon dioxide gas in the gas tank 60 is supplied from the valve 64 to the geed gas conduit 20 after pressure reductions by the first and second regulators 62 and 63, delivering the carbon dioxide gas to the fluid feed valve 20 through the gas feed conduit 21. In case the fluid feed valve 20 is in a position to communicate the gas feed conduit 21 with the gas supply passage 18, the carbon dioxide gas is fed to a body cavity from the fore distal end of the insertion rod 11.

At this time, since the air pump 23 is deactivated, air is not supplied to the compressed air conduit 24 nor to the gas feed conduit 21 to mix into carbon dioxide gas. As described above, simply by depressing or turning on the power switch 68 of the gas feeder 3, a supply of carbon dioxide gas is started while suspending an air supply. That is, in switching a gas supply, there is no need in particular for a switching action exclusively to turn off the air pump 23. Besides, the automatic turn-off of the air motor 23 contributes to prevent noises and unnecessary power consumption which would result from redundant operation of the air pump 23.

In this instance, the first communication line 81 which connects the light source 2 with the processor 4 has been used in existing systems for communication between the light source 2 and the processor 4. An iris control circuit (not shown) which is provided in the light source 2 is controlled from the processor 4 to adjust the light intensity. Therefore, the first communication line 81 is provided at least for the iris control. On the other hand, the gas feeder 3 should be connectible to various external units, and for this purpose a connection interface is provided on the gas feeder 3. Utilizing a connection interface of this sort, the gas feeder 3 is connected with the processor 4 by the second communication line 82.

As mentioned above, transmissions of information between the processor 4 and the light source 2 can be made by the use of the first communication line 81 which already exists. Therefore, there is no need for specially making a new communication line. However, it becomes necessary to introduce a new communication line for the second communication line 82 which connects the gas feeder 3 with the processor 4. Considering various controls which become necessary between the processor 4 and the gas feeder 3, the second communication line 82 can be utilized for transfer of necessary information.

The controller 80 is adapted to turn off the air pump 23 of the light source 2 when the power switch of the gas feeder 3 is turned on, so that it is connected to processor 4 in addition to the light source 2 and the gas feeder 3. For example, upon receipt of a signal indicative of turning-on of the power switch (a switch-on signal) through the second communication line 82 from the gas controller 61, the controller 80 outputs this switch-on signal to the light source controller 50. At this time, for example, the switch-on signal is superposed on an iris control signal. In this instance, the controller 80 is required to perform a signal superposing operation.

The processors 4 is fundamentally a means for processing video signals of endoscopically captured picture images, and for this purpose includes a CPU which is capable of complicate processing operations. Accordingly, it is possible to utilize part of functions of the CPU for the above-described control by the controller 80. In the particular embodiment shown, the controller 80 is connected to light source controller 50 and the gas controller 61. However, it is also possible to connect the controller 80 directly to the power switch 68 of the gas feeder 3 and the air pump 23 for the purpose of performing the above-described control. In this case, the control is complicated to some extent but can be easily executed by the use of the processing means. The control by the gas feed system of the invention can be realized without adding special control functions to the light source 2 and the gas feeder 3.

By the way, in the foregoing exemplary embodiment, the valve 64 is opened and closed in interlinked relation with on and off of the power switch 68. Therefore, a start of a carbon dioxide gas supply is detected from a switch-on action on the power switch 68. Of course, arrangements may be made to detect a start of a carbon dioxide gas supply by other methods. As shown in FIG. 3, a supply of carbon dioxide gas is started by opening the valve 64. Therefore, it is possible to recognize a start of a carbon dioxide gas supply by checking for opening of the valve 63 by the gas controller 61. By so doing, a start of a carbon dioxide gas supply can be recognized as soon as the valve 64 is opened by a manual operation on the valve switch 71.

Now, described below is a control of the gas feeder 3 according to residual gas content in the tank 60. The internal pressure of the gas tank 60 is constantly checked up by the first manometer 66, and the detected readings in residual gas pressure are output from the first manometer 66 to the gas controller 61. On the basis of a detected pressure reading (residual gas pressure in the gas tank 60), a residual gas content in the gas tank 60 is detected by the gas controller 61. A value (a threshold value) of a minimum necessary gas content for supply to a body cavity is preset in the gas controller 61 for comparison with a detected residual gas content. In this regard, arrangements may be made to compare gas pressures instead of gas contents.

If the residual gas content drops below the threshold value, it becomes difficult to inflate a body cavity to a sufficient degree by a carbon dioxide gas supply or to apply a sufficient pressure to the liquid surface in the liquid feed tank 25. In such a case, the gas controller 61 closes the valve 64 to suspend a carbon dioxide gas supply. At the same time, the suspension of a gas supply is notified to the controller 80 through the second communication line 2, whereupon the controller 80 controls the light source controller to turn on the air pump 23 to start an air supply. As a result, the air pump 23 is put in operation for an air supply.

Thus, as soon as the residual gas content in the tank 60 drops below a minimum necessary gas content during an endoscopic examination or treatment, the gas supply is automatically switched from carbon dioxide to air. Since the residual content of carbon dioxide gas in the tank 60 is limited, the gas supply is switched from carbon dioxide gas to air when the residual gas content drops below a predetermined threshold value, utilizing air which has abundantly unlimited source. That is to say, carbon dioxide gas is used as a main gas source while air is used as a supplementary gas source. Accordingly, it becomes possible to continue an endoscopic examination or treatment in an ordinary manner without facing a total shutdown of gas supply.

By the way, normally the fluid feed valve 20 is held in a closed position. Despite an increase in output pressure, the air pump 23 acts to further increase the output pressure. Therefore, the pressure can build up to an excessively high level by a continued operation of the air pump 23. To cope with such situations, the air pump 23 is provided with a relief mechanism 53 with a function of relieving excessively high pressures. More specifically, the relief mechanism 55 is adapted to relieve an excessively pressure when the pump pressure of the air pump 23 exceeds a predetermined relief point (a predetermined upper limit). Accordingly, while the fluid feed valve 20 is held in a closed position, the air pump 23 is put in operation at a maximum output pressure.

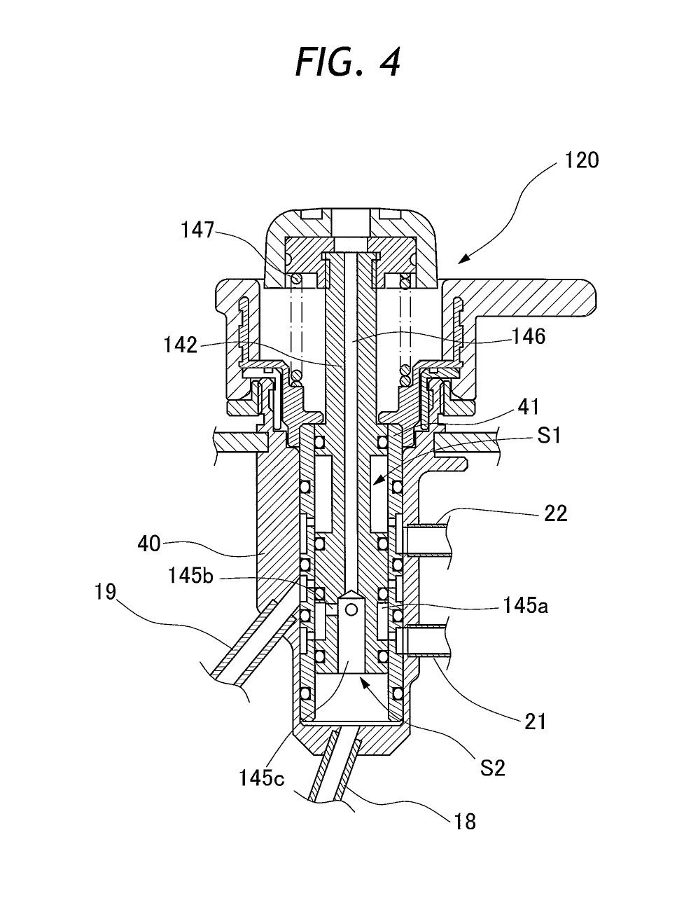

In this instance, as shown in FIG. 2, the fluid feed valve 20 is of a closed-to-atmosphere type which is blocked against communication with the atmosphere. However, in place of the fluid feed valve 20 of FIG. 2 (a fluid feed valve which is blocked against communication with the atmosphere), there may be employed an open-to-atmosphere type fluid feed valve 120 (a fluid feed valve which is communicable with the atmosphere) having an atmospheric communication passage as shown in FIG. 4. The fluid feed valve 120 of FIG. 4 is built into the manipulating head assembly 12 in the same manner as the fluid feed valve 20, including the valve casing 40, valve guide 41, gas supply passage 18, gas feed conduit 21, liquid supply passage 19 and liquid feed conduit 22, but employs a different valve member 142 in the valve guide 41 in place of the valve member 42 of FIG. 2.

In the valve position shown in the drawing, a second annular passage 145a at a second switching portion S2 of the valve member 142 is communicated with the gas supply passage 18 and the gas feed conduit 21 through a communication hole 145b and an open passage 145c. At the same time, it is opened to the atmosphere through an open passage 146 formed axially through the valve member 142 and the switch button 143. Therefore, the air pump 23 can be put in operation substantially in an idling state. If an outer end of the open passage 146 is blocked by putting a finger on the switch button 143, a pressure increase occurs at the second switch portion S2 between the gas supply passage 18 and the gas feed conduit 21 to supply air toward the gas supply passage 18. This is a gas feed position of the fluid feed valve 120. As the valve member 142 is further pushed down toward a liquid feed position against a biasing force of a return spring 147, the communication between the gas supply passage 18 and the gas feed conduit 21 is blocked, and instead the liquid supply passage 19 and the liquid feed conduit 22 are brought into communication with each other.

In this manner, in use, the valve member 142 of the fluid feed valve 120 is normally retained in an upper initial position under the influence of the basing force of the return spring 147, and can be switched to a gas feed position and a liquid feed position by depressing the switch button 143 against the action of the return spring 147 alone. Thus, in this case unlike the fluid feed valve 20 of FIG. 2, the valve member 142 which is retained in an upper initial position by the biasing action of the return spring 147 is not switched stepwise to a gas feed position and a liquid feed position firstly against the biasing force of the return spring 147 and secondly against a combined biasing force of the return spring 147 and a second biasing spring 49. That is to say, in this case, it is necessary to apply the biasing force of the return spring 147 to the valve member 142 there is no need for providing the second biasing spring 49 and the spring holder 48.

Thus, the closed-to-atmosphere type fluid feed valve 20 can be changed to the open-to-atmosphere type fluid feed valve 120 by replacing the valve member 47 in the valve guide 41 by the valve member 147. When the open-to-atmosphere type valve 120 is mounted, carbon dioxide gas is constantly released to the atmosphere through the open passage 146. Therefore, no matter whether the valve 120 is in a gas feed position or liquid feed position, carbon dioxide gas is wastefully consumed. However, depending upon familiarity with manipulative control operations, an operator can turn off a gas supply to prevent wasteful consumption of carbon dioxide gas through the open-to-atmosphere type fluid feed valve 120.

On the other hand, in case an operator prefers use of carbon dioxide gas, the closed-to-atmosphere type fluid feed valve 20 is mounted in position thereby to prevent wasteful consumption of carbon dioxide gas. Thus, depending upon whether or not an operator prefers use of carbon dioxide gas, a suitable type of fluid feed valve can be replaceably set on the endoscope to create optimum conditions by a supply of a selected gas.

Further, the light source 2 and the gas feeder 3, which are basically built as separate units, can be integrated into one assembly unit if desired. No matter whether the light source 2 and the gas feeder 3 are built as separate units or as one assembly unit, they constitute and function as part of the endoscopic gaseous material feed system of the invention. Therefore, arrangements may be made to power on the light source 2 and the gas feeder 3 when a power switch (not shown) of the gaseous material feed system is turned on to start the respective components of the system.

As described above, recently there is a trend toward using carbon dioxide gas as a feed gas source in an endoscopic gaseous material feed system. Therefore, it is desirable to control the gas controller 61 of the gas feeder 3 to open the valve 64 for a supply of carbon dioxide gas at the time when a power switch of the system is turned on, while holding the air pump 23 of the light source 2 in a deactivated state. By so arranging, carbon dioxide gas is preferentially supplied upon starting the system.

What is claimed is:

1. An endoscopic gaseous material feed system, comprising:
a gas feeder having a gas tank packed with carbon dioxide gas;
a light source incorporating an air pump for air supply;
an endoscope internally formed with a gaseous material passage to supply carbon dioxide gas or air to a body cavity of a patient or examinee; and
a controller adapted to put said air pump of said light source in a deactivated state upon detection of a start of a carbon dioxide gas supply from said gas feeder.

2. An endoscopic gaseous material feed system as set forth in claim 1, further comprising:
a processor adapted to process video signals of endoscopically captured picture images on the basis of electric signals from a solid-state image sensor mounted on a distal end portion of said endoscope;
said controller being provided in said processor electrically connected with said light source and said gas feeder.

3. An endoscopic gaseous material feed system as set forth in claim 2, wherein said controller is adapted to suspend a supply of carbon dioxide gas from said gas feeder as soon as a residual gas content in said gas tank drops below a predetermined threshold value, while activating said air pump to start an air supply from said light source.

4. An endoscopic gaseous material feed system as set forth in claim 3, wherein, on a manipulating head assembly, said endoscope is provided with a gas feed conduit at a junction of a carbon dioxide gas supply route from said gas feeder and an air supply route from said light source, a liquid feed conduit adopted as a passage of a liquid delivered from a liquid feed tank under pressure applied by said carbon dioxide gas or air on a liquid surface in said liquid feed tank, and a fluid feed valve adapted to selectively take in either a feed gas delivered under pressure through said gas feed conduit or a feed liquid delivered under pressure through said liquid feed conduit;
said fluid feed valve being adapted to permit replacement of an open-to-atmosphere type fluid feed valve in communication with the atmosphere for release of a gas in said gas feed conduit, by a closed-to-atmosphere type fluid feed valve blocking communication of said gas feed conduit with the atmosphere, or vice versa.

5. An endoscopic gaseous material feed system as set forth in claim 2 or 3, further comprising a relief mechanism to relieve pump pressure of said air pump to the atmosphere when reached beyond a predetermined upper limit.

6. An endoscopic gaseous material feed system as set forth in claim 2 or 3, further comprising a main power switch to turn on and off said gas feeder and said light source together,
said controller being adapted to start a carbon dioxide gas supply from said gas feeder when said main power switch is turned on, while suspending an air supply from said air pump.
